(19) United States
(12) Patent Application Publication
(10) Pub. No.: US 2025/0230416 A1
(43) Pub. Date: Jul. 17, 2025

(54) MULTI DONOR STEM CELL COMPOSITIONS AND METHODS OF MAKING SAME

(71) Applicant: Children's Hospital Medical Center, Cincinnati, OH (US)

(72) Inventors: Takanori Takebe, Cincinnati, OH (US); Masaki Kimura, Cincinnati, OH (US); Ran-Ran Zhang, Cincinnati, OH (US)

(73) Assignee: Children's Hospital Medical Center, Cincinnati, OH (US)

(21) Appl. No.: 19/058,609

(22) Filed: Feb. 20, 2025

Related U.S. Application Data
(63) Continuation of application No. 16/603,609, filed on Oct. 8, 2019, now Pat. No. 12,281,334.
(60) Provisional application No. 62/517,414, filed on Jun. 9, 2017.
(60) Provisional application No. 62/485,562, filed on Apr. 14, 2017.

(51) Int. Cl. C12N 5/071; C12N 5/074; G01N 33/50

(52) U.S. Cl. CPC C12N 5/0697; C12N 5/0679; C12N 5/0696; G01N 33/5008; C12N 2500/02; C12N 2501/40

(57) ABSTRACT

Disclosed are compositions, in particular, organoid compositions, derived from more than one donor cell. Further disclosed are methods of making compositions, for example, organoid compositions, that comprise a differentiated cell population derived from more than one donor cell. Donor cells may include, for example, a precursor cell such as an embryonic stem cell or other precursor cell. The disclosed methods use synchronization conditions to produce a synchronized pooled-precursor cell population, which may then be differentiated into an organoid composition. Methods of using the compositions are also disclosed.

### CROSS-REFERENCE TO RELATED APPLICATIONS

This application is a Continuation Application of U.S. Nonprovisional Ser. No. 16/603,609, filed Oct. 8, 2019, which is a 371 National Stage Application of PCT/US18/27585, filed Apr. 13, 2018, which claims priority to and benefit of U.S. Provisional Application 62/485,562 filed Apr. 14, 2017, and U.S. Provisional Application Ser. No. 62/517,414 filed Jun. 9, 2017. The contents of each are incorporated in their entirety for all purposes.

### BACKGROUND

Efficient methods for the study of large populations are an unmet need in the art. For example, the study of safety and efficacy of a potential new drug or treatment in a population is a requirement for clinical trials for drug approval. Preclinical detection of risk compounds for drug induced liver injury (DILI) remain a significant challenge in drug development, highlighting a need for a predictive human system. That is, clinical trials are costly and time consuming, and many drugs fail for toxicity and/or lack of efficacy-often after much investment in clinical trials has been made. Early determination of toxicity (in particular, drug induced hepatotoxicity) or efficacy using in vitro research tools that are representative of a population could shorten the time to development and/or avoid unnecessary injury to clinical trial participants, and/or decrease costs associated with bringing drugs to market. A viable in vitro platform to screen a population cohort for drug safety and efficacy studies would therefore be highly desirable. Further desirable is a screen useful for early detection in vulnerable populations, such as individuals having compromised liver function.

In addition, there exists many disease states for which a genetic cause is unknown—there is therefore a need for improved methods for identifying the genetic basis for disease states that appear in a population. Human induced pluripotent stem cells (hiPSC) and other precursor cells provide a promising opportunity to establish human-specific models of various disease for drug development. Population-scale iPSC reprogramming initiatives may allow for the study of any imaginable population cohort for disease phenotypes, drug safety, and efficacy. However, one to one comparison of each individual stem cell is inefficient and completely unrealistic due to both time and cost considerations. Therefore, a viable platform to screen a population phenotype in a pooled sample of various donor derived precursor cells would be highly desirable. While methods of making differentiated cell populations and organoid compositions from precursor cells have been described in the art, directed differentiation of cell populations and/or organoids derived from more than one donor has not been accomplished due to the need to synchronize growth and differentiation of cells from different donors. The instant disclosure seeks to address one or more of the aforementioned needs in the art.

### BRIEF SUMMARY

Disclosed are compositions, in particular, organoid compositions, derived from more than one donor cell, thus allowing for cellular representation of one or more donors, or, in some cases, a population of donors. Further disclosed are methods of making such compositions, for example, organoid compositions that comprise a differentiated cell population derived from more than one donor cell that may represent a desired population of donors. Donor cells useful for deriving the disclosed organoid compositions may include, for example, a precursor cell such as an embryonic stem cell or other precursor cell. The disclosed methods use synchronization conditions to produce a synchronized pooled-precursor cell population, which may then be differentiated via directed differentiation into an organoid composition. Methods of using the compositions are also disclosed.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts an analysis of cell proliferation and metabolism to examine donor-dependent variation. Top: Cell proliferation assay, which shows the difference in the proliferative property of iPSCs. iPSCs were plated at 5×10 4 cells and cell numbers were count. Bottom: Metabolomics analysis showing the difference in metabolism of iPSCs. PCA scores were plotted using the 30 metabolites assigned buckets.

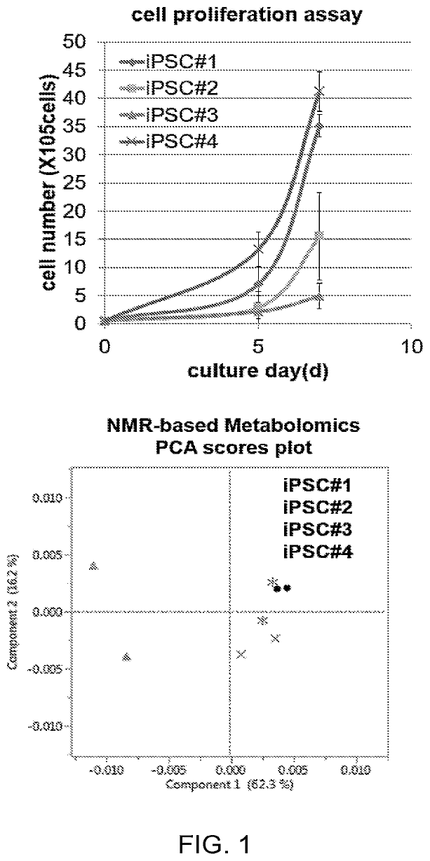

FIG. 2 shows synchronizing growth kinetic variabilities by hypoxic culture condition. Hypoxic culture promoted cell synchronization by regulating cell growth kinetics. Top: Morphology of cells in hypoxic culture condition. Bottom left: Cell proliferation in each oxygen conditioning. Cell proliferation of donor-dependent variation was canceled in the 2% hypoxic condition. Bottom right: Cell proliferation synchronization analysis. The difference in the growth rate of each cell was plotted. Synchronization index (SYNDEX) is the reciprocal of donor difference standard deviation.

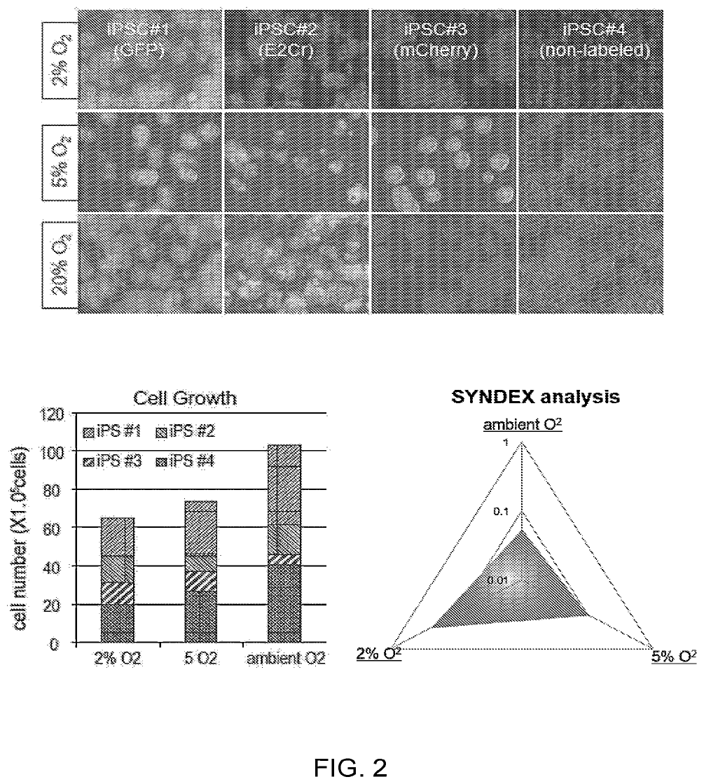

FIG. 3 depicts synchronizing growth kinetic variabilities by compound cocktail treatment. Compound cocktail treatment was supported cell synchronization by combining with hypoxic culture. Top: Cell proliferation in inhibitor treatment. Bottom: Cell proliferation synchronization analysis.

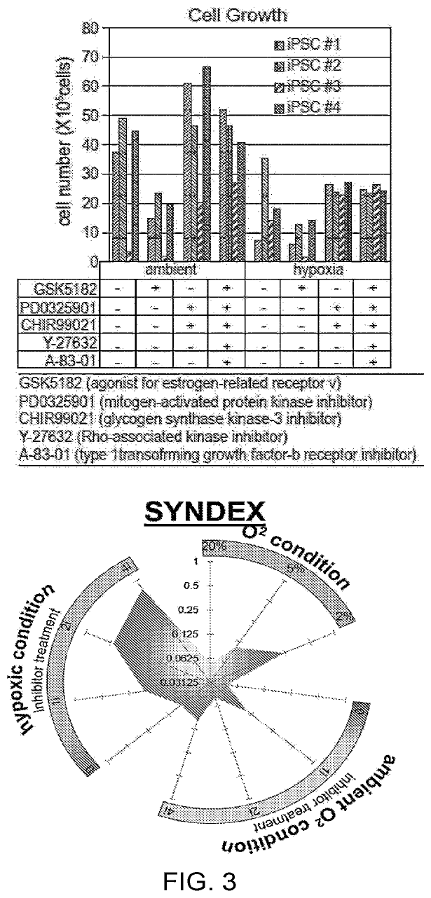

FIG. 4 shows pooling culture experiment of iPSC derived from multi donors. Multiple iPSCs with fluorescent labels were mixed and cultured in a hypoxic state. As a result of fluorescence microscopic observation and FACS population analysis, hypoxic condition enabled synchronization and maintenance in each cell population on the same dish.

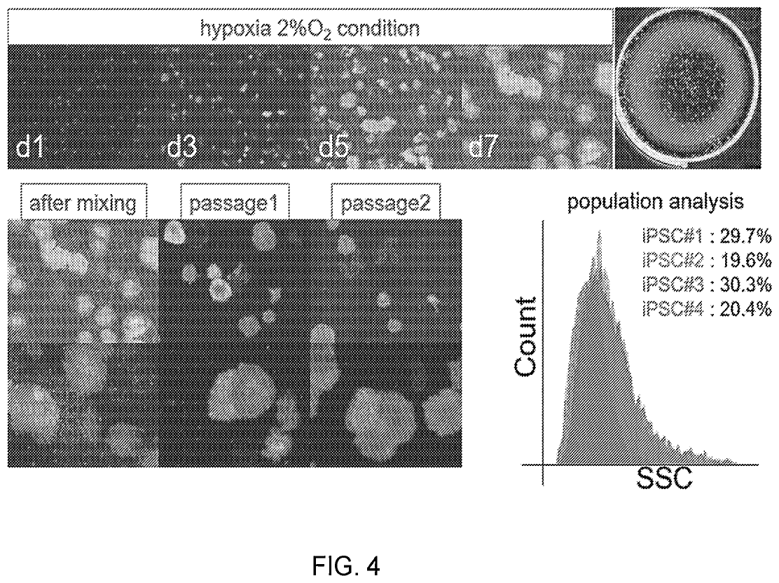

FIG. 5 shows parallel differentiation induction of various cell types from iPSC pool. In order to confirm the pluripotency of pooled iPSC, Applicant attempted to induce multiple lineage including definitive endoderm, mesoderm, posterior foregut, endothelial cells, hepatocytes, and liver organoids. As a result of gene expression, FACS analysis and specific functional analysis, it was demonstrated that pooled iPSC had multi lineage differentiation potential.

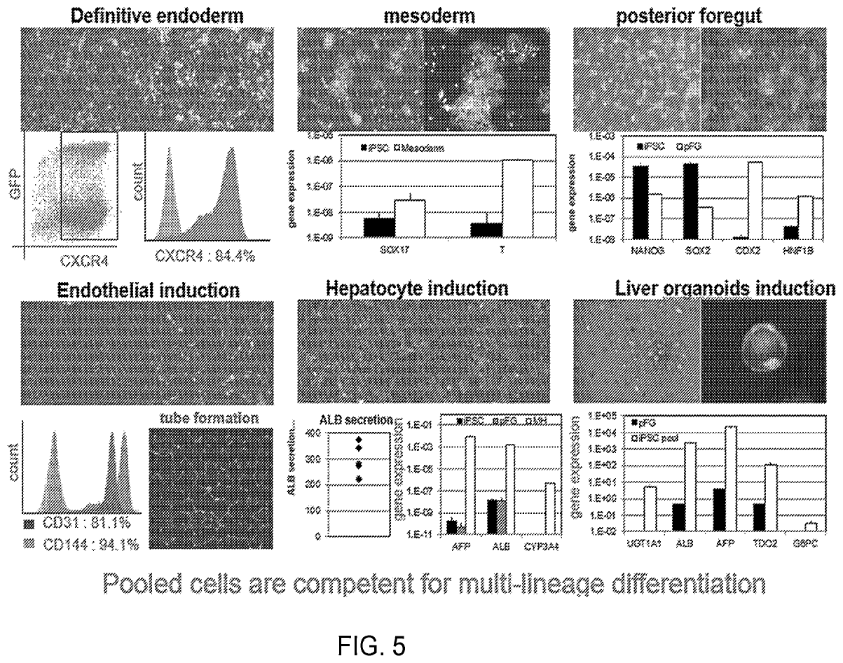

FIG. 6 shows iPSC pooling overcomes differentiation propensity to endothelial cells. In order to confirm the effect of iPSC pooling, Applicant attempted to induce endothelial cell induction using iPSC lines with low endothelial induction efficiency. Top: The morphology of endothelial cell induction in each cell line and pooled cell line. Bottom: FACS analysis of endothelial specific marker expression in each cell line and pooled cell line. The endothelial differentiation efficiency was significantly improved in pooled iPSC culture.

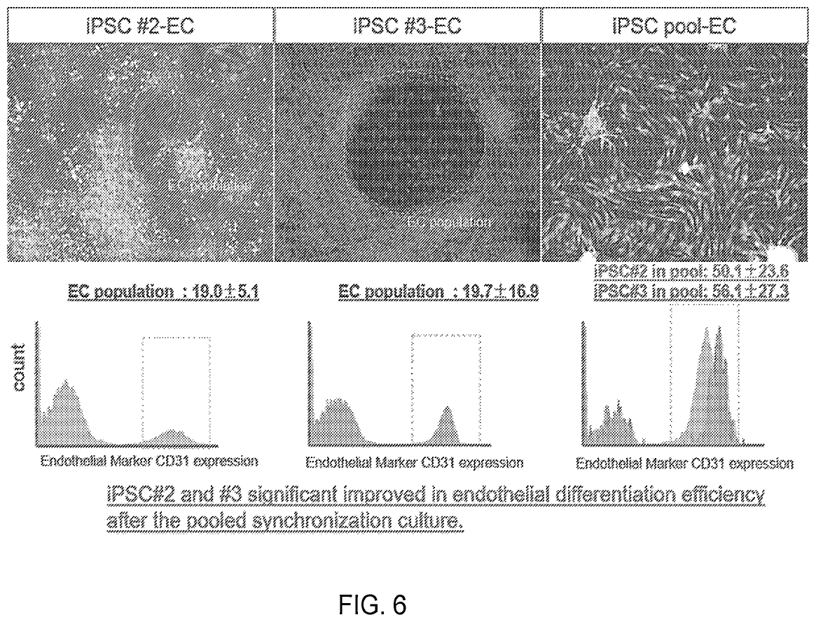

FIG. 7 shows confirmation of neural differentiation induction efficiency of iPSC established from multiple donor-derived fibroblast. Neurite outgrowth was observed on day 23 of induction of neuronal differentiation of multiple donor-derived iPSC

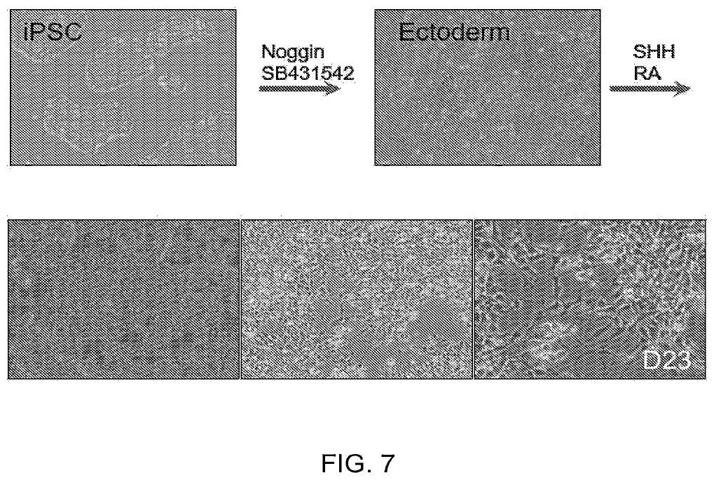

FIG. 8 shows confirmation of neural differentiation induction efficiency of iPSC established from multiple donor-derived fibroblast. Expression of neuron-specific genes were significantly increased at day 23 of differentiation induction.

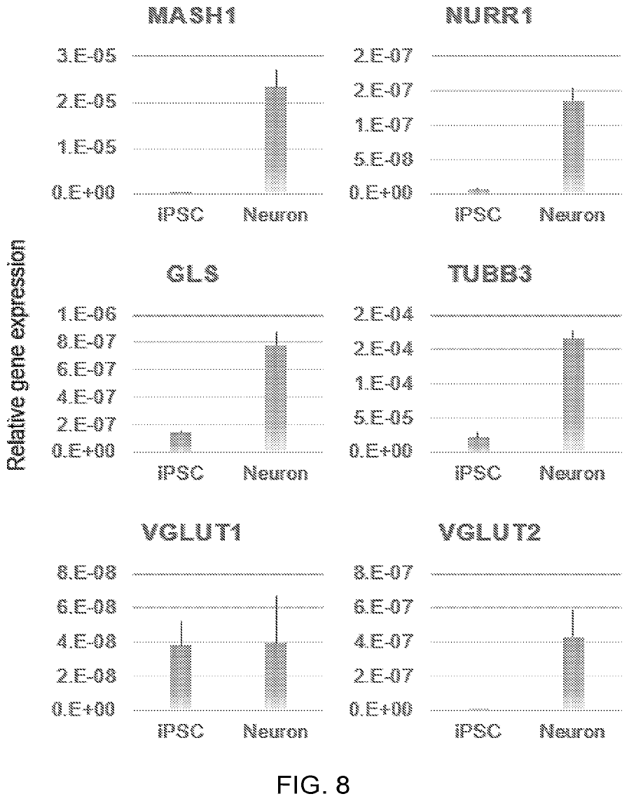

FIG. 9 shows confirmation of neural differentiation induction efficiency of iPSC established from multiple donor-derived fibroblast. The neuron specific markers TUJ 1, PAX 6 and TH were expressed at day 23 of differentiation.

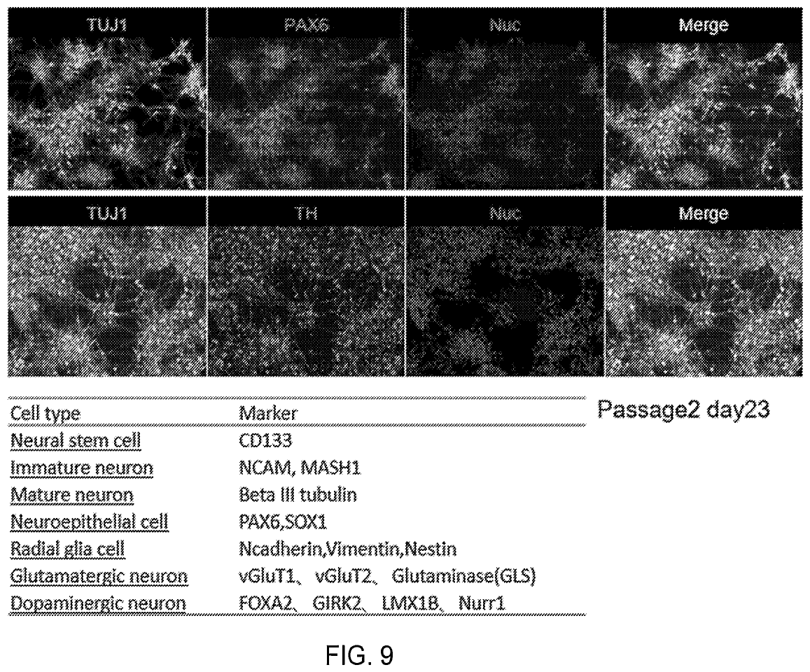

FIG. 10 shows confirmation of neural differentiation induction efficiency of iPSC established from multiple donor-derived fibroblast. The neuron specific markers TUJ 1, PAX 6 and TH were expressed at day 23 of differentiation.

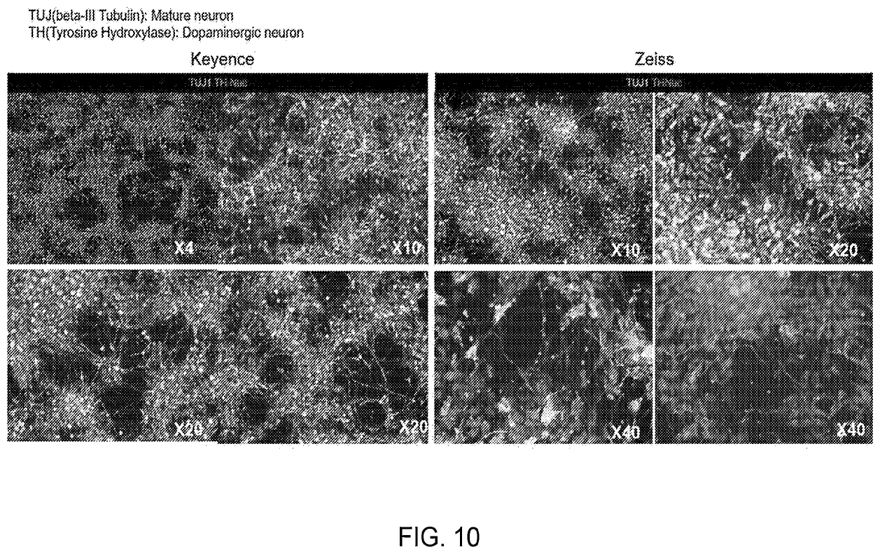

FIG. 11 shows establishment of freezing method of iPSC derived foregut cell. To establish the freezing method of iPSC-derived foregut cell, we attempted several cryopreservation conditions. As a result of freezing and thawing, the survival rate of single cell isolated cells was significantly higher than the cluster condition cell.

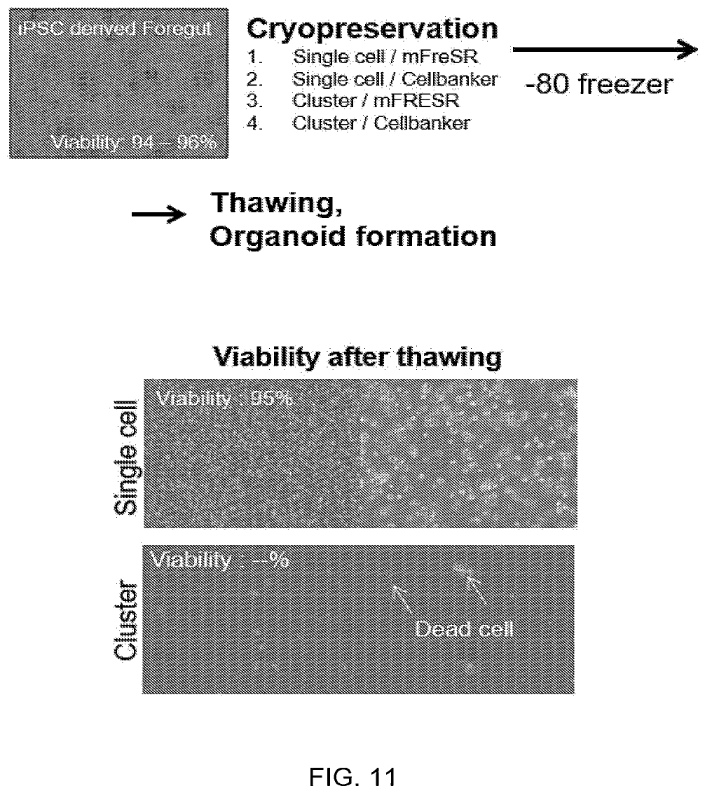

FIG. 12 shows establishment of freezing method of iPSC derived foregut cell. To establish the freezing method of iPSC-derived foregut cell, Applicant attempted to generate liver organoids using frozen cells. As a result of organoid formation, the organoid formation efficiency of single cell isolated cells was significantly higher than the cluster condition cell.

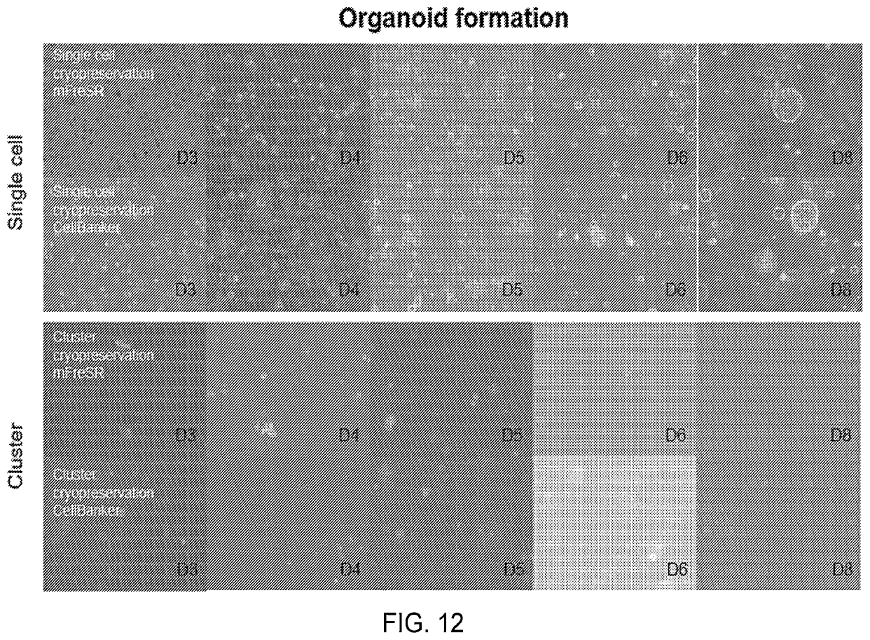

FIG. 13 shows clonal liver organoid formation from frozen foregut pool. Applicant developed a method to form liver organoids from frozen foregut pools. Organoid formation was performed by plating 5000 cells to 40000 cells in 10 μl Matrigel drops, respectively. The pooled cells were used fluorescently labeled foregut cells (Green, Red, blue) and non-labeled foregut cells.

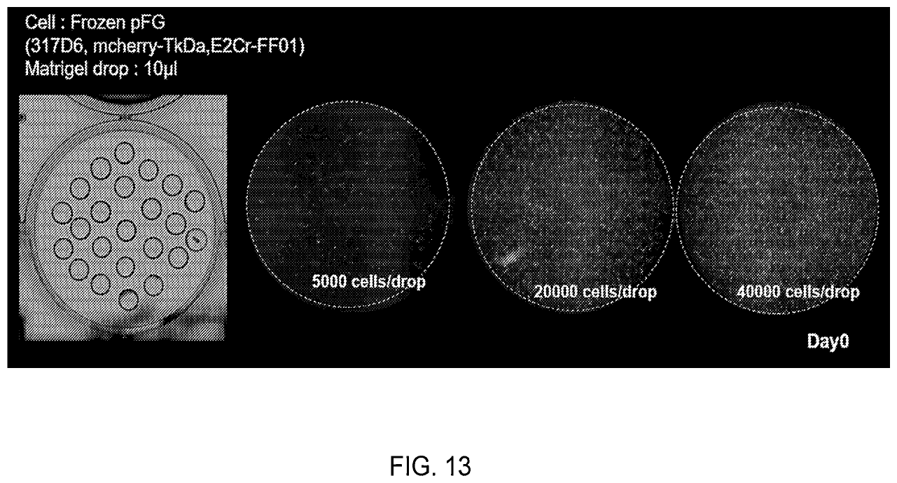

FIG. 14 shows clonal liver organoid formation from iPSC pool. Organoids were successfully formed under the conditions of 20000 cells and 40000 cells. In addition, it was confirmed that the formed organoid was monochromatic and derived from a single donor.

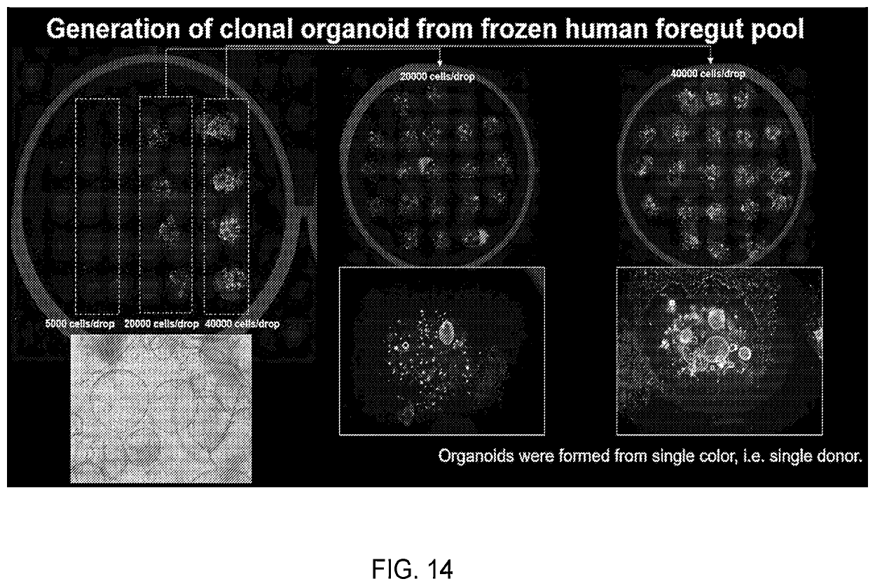

FIG. 15 shows live imaging-based detection of iron accumulation in hepatocyte and organoids from pool. The pooled cells were used fluorescently labeled foregut cells (Green, blue) and non-labeled foregut cells. For the detection of iron accumulation, a red fluorescent reagent FeRhoNox-1 which reacts specifically with iron ions was used.

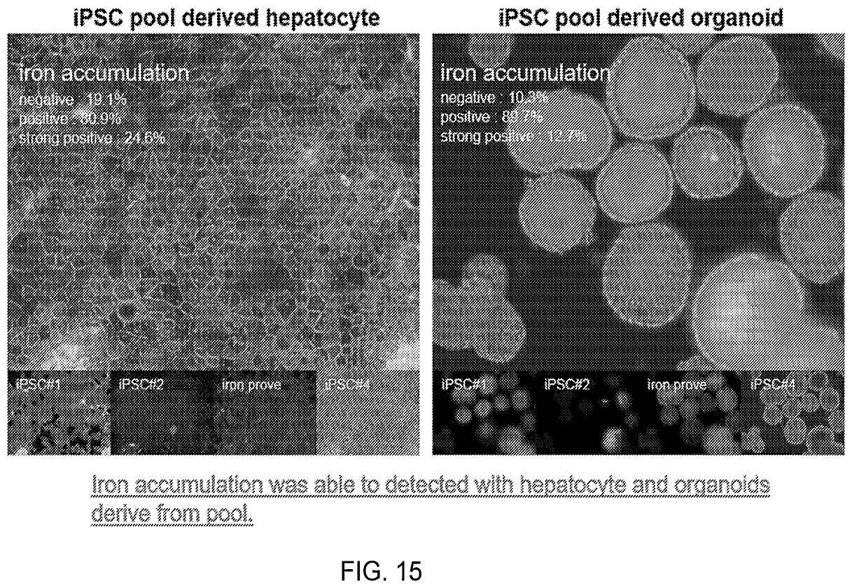

FIG. 16 shows live imaging-based detection of iron accumulation in hepatocyte and organoids from pool. Two types of fluorescently labeled cells and diseased cells derived from hemochromatosis were used as a positive control for iron accumulation to prepare pooled hepatocytes. Quantitative results of iron accumulation show that it was significantly higher in hemochromatosis derived cells.

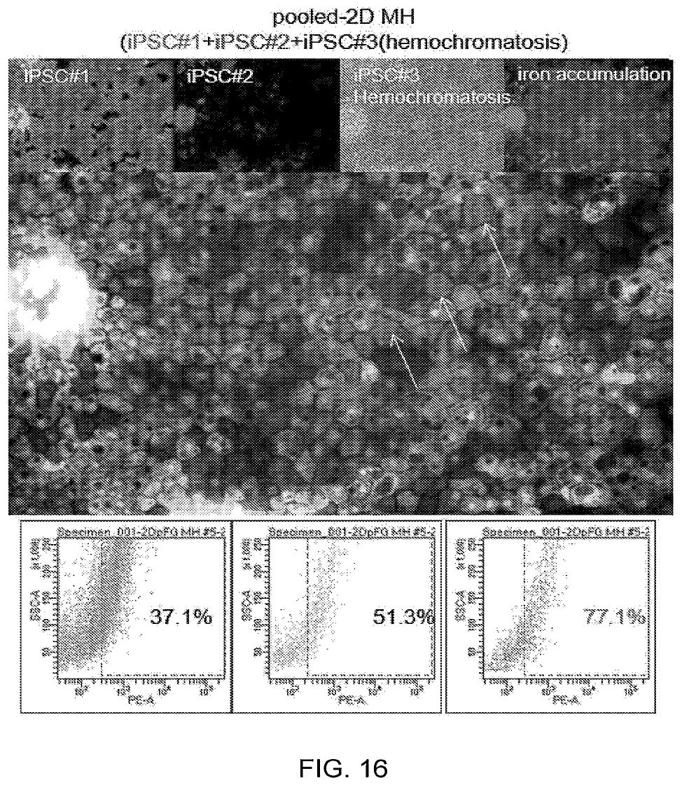

FIG. 17 shows live imaging-based detection of iron accumulation in hepatocyte and organoids from pool. As with 2D hepatocyte experiment, two types of fluorescently labeled cells and hemochromatosis derived cells were used to prepare pooled liver organoids. Quantitative results of iron accumulation were significantly higher in cells derived from hemochromatosis.

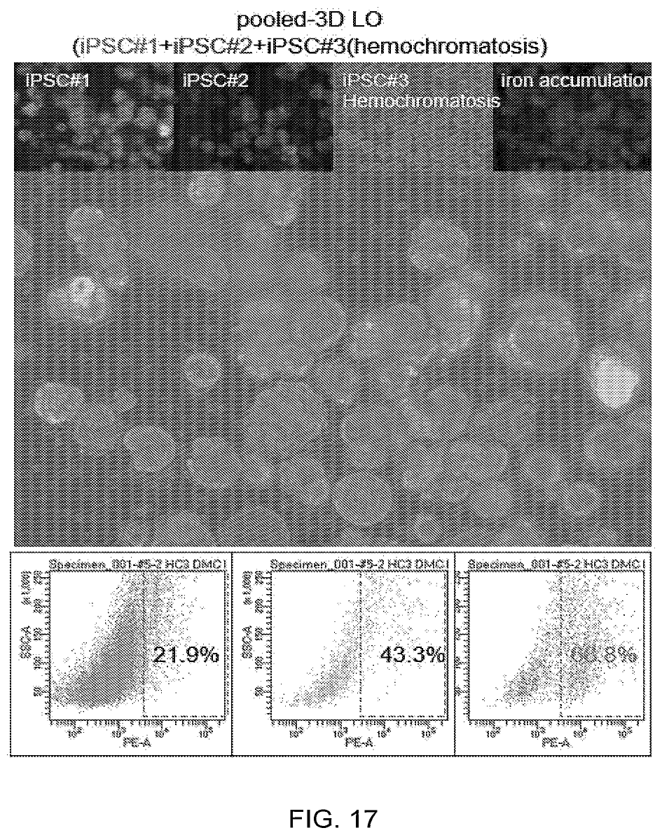

FIG. 18 shows development of non-invasive donor decoding method in pooled stem cell culture by environmental DNA and a conceptual image of donor identification using donor specific SNPs PCR.

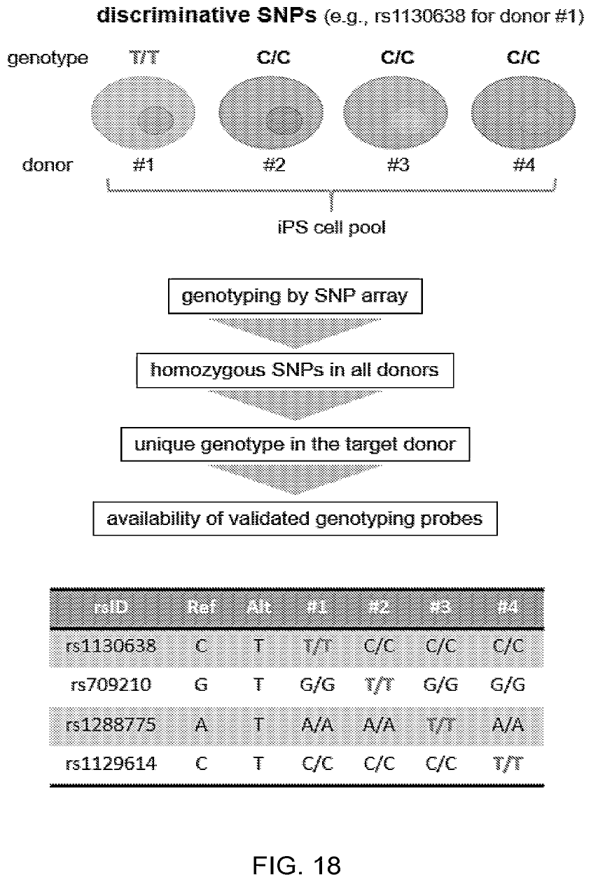

FIG. 19 shows development of non-invasive donor decoding method in pooled stem cell culture by environmental DNA and a conceptual image of donor identification using donor specific SNPs PCR.

FIG. 20 shows donor ratio estimation from culture medium in iPSC pool. Applicant attempted to estimate the donor ratio of iPSC pool using environmental DNA (“eDNA”) contained in the culture supernatant. As a result, the cell ratio was successfully identified by PCR of eDNA in culture medium. The eDNA ratio in the culture medium was also correlated with attached cells.

FIG. 21 shows donor ratio estimation from culture medium in iPSC pool. Applicant also attempted to estimate the donor ratio of pooled hepatocyte and liver organoid by eDNA. As a result, the cell ratio was successfully identified by PCR of eDNA in the medium.

FIG. 22. Donor ratio change monitored by gDNA in the culture medium. To confirm the eDNA donor identification, Applicant monitored cell ratio change by drug selection. Two types of fluorescently labeled cells and puromycin resistant cells were used as a positive control. As a result of drug selection, non-resistant iPSCs were disappeared on the second day of treatment. The result of eDNA donor identification PCR showed that the ratio of nonresistant cells decreased from the second day and only resistant cells were detected on the fourth day.

FIG. 23. Drop-out assay using induced hepatocytes treated with oleic acid and troglitazone. Drug screening assays for fat accumulation and drug treatment were performed using hepatocytes prepared from iPSC pools. Eleven iPSCs with different donors were used for iPSC derived hepatocyte pool. eDNA based drop-out assay confirmed cells with high fat accumulation risk donor were significantly more sensitive to drug induced hepatotoxicity than healthy donor derived cells

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, for example within 5-fold, or within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

As used herein, the term “pluripotent stem cells (PSCs)” encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes. hiPSC refers to human iPSCs. In some embodiments, iPSCs may be derived by transfection of certain stem cell-associated genes into non-pluripotent cells, such as adult fibroblasts. Transfection may be achieved through viral vectors, such as retroviruses. Transfected genes may include the master transcriptional regulators Oct-3/4 (Pouf51) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some embodiments, a retroviral system is used to transform human fibroblasts intopluripotent stem cells using four pivotal genes: Oct3/4, Sox2, Klf4, and c-Myc. In alternative embodiments, a lentiviral system is used to transform somatic cells with OCT4, SOX2, NANOG, and LIN28. Genes whose expression are induced in iPSCs include but are not limited to Oct-3/4 (e.g., Pou5fl); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, and LIN28.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

Applicant has developed a system for synchronization of growth kinetic variabilities among multiple iPSC and expansion and differentiation of stem cell pool into specific cell types, which may be further developed into an organoid of a desired type, but which may comprise cells from more than one donor. In developmental biology, cellular differentiation is the process by which a less specialized cell becomes a more specialized cell type. As used herein, the term “directed differentiation”, or more generically, “differentiation” in situations in which external factors are applied to induce or control differentiation, may describe a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In one aspect, a composition comprising a differentiated cell population derived from more than one precursor cell is disclosed, wherein said differentiated cell population is synchronized in development—i.e., the cells are differentiated to a desired cellular stage at substantially the same point in time, for example, via mitotically promoting slow cycling stem cells and/or inhibiting fast cycling stem cells. The synchronized, differentiated cell population may comprise cells from more than one individual. In one aspect, the synchronized, differentiated cell population may be selected from definitive endoderm, mesoderm, exoderm, posterior foregut, endothelium, hepatocytes, or an organoid. The more than one individual may, in certain aspects, comprise a population of individuals with or without a disease of interest.

In some embodiments, stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present invention include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present invention include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UC01 (HSF1); UC06 (HSF6); WA01 (H1); WA07 (H7); WA09 (H9); WA13 (H13); WA14 (H14). More details on embryonic stem cells can be found in, for example, Thomson et al., 1998, “Embryonic Stem Cell Lines Derived from Human Blastocysts,” Science 282 (5391): 1145-1147; Andrews et al., 2005, “Embryonic stem(ES) cells and embryonal carcinoma (EC) cells: opposite sides of the same coin,” Biochem Soc Trans 33:1526-1530; Martin 1980, “Teratocarcinomas and mammalian embryogenesis,” Science 209 (4458): 768-776; Evans and Kaufman, 1981, “Establishment in culture of pluripotent cells from mouse embryos,” Nature 292 (5819): 154-156; Klimanskaya et al., 2005, “Human embryonic stem cells derived without feeder cells,” Lancet 365 (9471): 1636-1641; each of which is hereby incorporated herein in its entirety.

In one aspect, the differentiated cell population may comprise an organoid, the organoid being derived from a precursor cell from more than one donor, or more than one to about 1000 donors, or from about 100 donors to about 500 donors. The organoid may be any organoid known to one of ordinary skill in the art, and may include, for example, a liver organoid, a gastric organoid, an intestinal organoid, a brain organoid, a pulmonary organoid, a bone organoid, a cartilage organoid, a bladder organoid, a blood vessel organoid, an endocrine organoid (such as thyroid, pituitary, adrenal, or the like), a sensory organoid (ear, eye, taste, tongue, or the like).

Methods of developing organoid tissues have been disclosed in the art. The disclosed methods allow for development of an organoid derived from a precursor cell, such as an iPSC, or embryonic stem cell (ES cell), from more than one individual. The resulting “pooled organoid” combines cells from more than one donor, providing unique properties to and allowing for novel uses for the resulting organoid. Methods of making various organoids are known in the art and are described in, for example, the following references: Dekkers, J. F., Wiegerinck, C. L., de Jonge, H. R., Bronsveld, I., Janssens, H. M., de Winter-de Groot, K. M., Brandsma, A. M., de Jong, N. W., Bijvelds, M. J., Scholte, B. J., et al. (2013). A functional CFTR assay using primary cystic fibrosis intestinal organoids. Nat Med 19, 939-945; Lancaster, M. A., and Knoblich, J. A. (2014). Organogenesis in a dish: modeling development and disease using organoid technologies. Science 345, 1247125; Nakamura, T., and Sato, T. (2018). Advancing Intestinal Organoid Technology Toward Regenerative Medicine. Cell Mol Gastroenterol Hepatol 5, 51-60; Sachs, N., de Ligt, J., Kopper, O., Gogola, E., Bounova, G., Weeber, F., Balgobind, A. V., Wind, K., Gracanin, A., Begthel, H., et al. (2018). A Living Biobank of Breast Cancer Organoids Captures Disease Heterogeneity. Cell 172, 373-386 e310; Sasai, Y. (2013). Cytosystems dynamics in self-organization of tissue architecture. Nature 493, 318-326; Takebe, T., Sekine, K., Enomura, M., Koike, H., Kimura, M., Ogaeri, T., Zhang, R. R., Ueno, Y., Zheng, Y. W., Koike, N., et al. (2013). Vascularized and functional human liver from an iPSC-derived organ bud transplant. Nature 499, 481-484; Takebe, T., Sekine, K., Kimura, M., Yoshizawa, E., Ayano, S., Koido, M., Funayama, S., Nakanishi, N., Hisai, T., Kobayashi, T., et al. (2017). Massive and Reproducible Production of Liver Buds Entirely from Human Pluripotent Stem Cells. Cell Rep 21, 2661-2670; Workman, M. J., Mahe, M. M., Trisno, S., Poling, H. M., Watson, C. L., Sundaram, N., Chang, C. F., Schiesser, J., Aubert, P., Stanley, E. G., et al. (2017). Engineered human pluripotent-stem-cell-derived intestinal tissues with a functional enteric nervous system. Nat Med 23, 49-59.

In one aspect, a method of making a composition comprising a differentiated cell population derived from a plurality of donors is disclosed. The differentiated cell population may comprise a population of cells derived from more than one donor, for example, from about 10 donors to about 1,000 donors or more, or from about 100 to about 500 donors. The method may comprise the steps of exposing a pooled-iPSC population to a synchronization condition for a period of time sufficient to produce a synchronized pooled-iPSC population; contacting the synchronized pooled-iPSC population with a “compound cocktail” sufficient to differentiate the synchronized pooled-iPSC population into a differentiated cell population. In one aspect, the compound cocktail may comprise a mitogen-activated protein kinase inhibitor (MEK/ERK pathway inhibitor), for example, PD0325901and a glycogen synthase kinase-3 inhibitor, for example, CHIR99021. In other aspects, the compound cocktail may further comprise an estrogen-related receptor gamma agonist, for example, GSK5182, a Rho-associated kinase inhibitor (Rho/ROCK pathway inhibitor), for example Y-27632 and a type 1 transforming growth factor-b receptor inhibitor, for example A-83-01. Exemplary concentrations include GSK5182; 5nM-50 μM, PD0325901; 1 nM-10 μM, CHIR99021; 3 nM-30 μM, A83-01; 0.5 nM-5 μM, Y27632; 10 nM-100 μM

In other aspects, the following agents may be used in lieu of or in addition to the aforementioned agents.

RHO/ROCK pathway inhibitor: Y-27632 dihydrochloride (R&D #1254)

GSK3 pathway inhibitors: CHIR 99021 (R&D #4423), Beryllium, Copper, Lithium, Mercury, Tungsten, 6-BIO, Dibromocantharelline, Hymenialdesine (Enzo Life Sciences), Indirubins, Meridianins, CT98014 (Axon Medchem), CT98023 (BOC Sciences), CT99021, TWS119, SB-216763, SB-41528, AR-A014418, AZD-1080, Alsterpaullone (Sigma), Cazpaullone (MedKoo), Kenpaullone (Sigma), Manzamine A (Enzo Life Sciences), Palinurine, Tricantine, TDZD-8, NP00111, NP031115, Tideglusib, HMK-32, L803-mts (Sigma), Ketamine (Sigma), or combinations thereof. Such agent may be present in an amount of from about 10nM to about 100 μM, or from about 100 nM to about 50 μM.

TGFB pathway inhibitors: Stemolecule A83-01 (Stemgent #47132), SD 208, LY2109761, SB525334, Pirfenidone, GW788388, RepSox, LY 2157299, LDN 193189 hydrochloride, SB 431542, Dorsomorphin, A 77-01, GW 788388, Dorsomorphin dihydrochloride, K 02288, SB 505124, LDN 212854 trihydrochloride, SB 525334, ITD 1, SIS3, or combinations thereof. Such agent may be present in an amount of from about 0.5 nM to about 5 μM, or from about 1 nM to about 2.5 μM.

MEK/ERK Pathway Inhibitors, PD0325901 (R&D #4192/10) (R&D #4192/10), Selumetinib (AZD6244), PD0325901, Trametinib (GSK1120212), U0126-EtOH, PD184352 (CI-1040), PD98059, BIX 02189, Pimasertib (AS-703026), BIX 02188, TAK-733, AZD8330, Binimetinib (MEK162, ARRY-162, ARRY-438162), PD318088, Honokiol, SL-327, Refametinib (RDEA119, Bay 86-9766), Myricetin, BI-847325, Cobimetinib (GDC-0973, RG7420), APS-2-79 HCl, GDC-0623, or combinations thereof. Such agent may be present in an amount of from about 1 nM to about 10 μM, or from about 10 nM to about 5 μM.

Estrogen-related receptor γ(ERRγ) agonists: GSK5182 (Aobious #AOB1629), AC 186, Daidzein, DPN, (R)-DPN, DY131, (S)-Equol, ERB 041, α-Estradiol, β-Estradiol, Estropipate, FERb 033, GSK 4716, 27-Hydroxycholesterol, Liquiritigenin, PPT, WAY 200070, XCT 790, or combinations thereof. Such agent may be present in an amount of from about 5 nM to about 50 μμM, or from about 10 nM to about 25 μM.

Source information: The following may be obtained, for example, from Selleck Chemical company: Indirubins, 6-BIO, CT99021, TWS119, SB-216763, SB-41528, AR-A014418, AZD-1080, TDZD-8, Tideglusib, SD 208, LY2109761, SB525334, Pirfenidone, GW788388, RepSox, LY 2157299, LDN 193189 hydrochloride, SB 431542, Dorsomorphin, A 77-01, GW 788388, Dorsomorphin dihydrochloride, K 02288, SB 505124, LDN 212854 trihydrochloride, SB 525334, ITD 1, SIS3, Selumetinib (AZD6244), Trametinib (GSK1120212), U0126-EtOH, PD184352 (CI-1040), PD98059, BIX 02189, Pimasertib (AS-703026), BIX 02188, TAK-733, AZD8330, Binimetinib (MEK16 2, ARRY-162, ARRY-438162), PD318088, Honokiol, SL-327, Refametinib (RDEA119, Bay 86-9766), Myricetin, BI-847325, Cobimetinib (GDC-0973, RG7420), APS-2-79 HCl GDC-0623, AC 186, Daidzein, DPN, (R)-DPN, DY131, (S)-Equol, ERB 041, α-Estradiol, β-Estradiol, Estropipate, FERb 033, GSK 4716, 27-Hydroxycholesterol, Liquiritigenin, PPT, WAY 200070, XCT 790.

In one aspect, the cells are in culture for at least four days to allow for synchronization of the mixed donor cell population. In other aspects, the amount of time for synchronization may be from about 1 to about 6 or about 2 to about 5 or about 4 days. For proliferative kinetics, eDNA based monitoring of non-labeled stem cell pools or image-based analysis of differently labeled stem cell pools can be used. In one aspect, the methods may be carried out for a period of time sufficient for the following markers of pluripotentcy to be expressed: NANOG, OCT4, SOX2, SSEA4 and TRA160. In one aspect, the synchronization condition may comprise a hypoxic culture condition, for example, wherein said hypoxic culture condition is between about 2% O₂ to about 10% O₂, or between about 2% to about 5%, or about 2%, or about 3%, or about 4%, or about 5%, or up to 6% or up to 7%, or up to about 8% O₂

In one aspect, a method of making a composition comprising a differentiated cell population from more than one donor is disclosed. The differentiated cell population may be derived from more than one individual, for example, from about 10 individuals to about 1,000 donors, or from about 100 to about 500 donors. The method may comprise the steps of exposing a pooled-endoderm population to a clonal condition for a period of time sufficient to produce a synchronized pooled-endoderm population; contacting said synchronized pooled-endoderm population with a compound cocktail sufficient to differentiate said synchronized pooled-endoderm population into a differentiated cell population, wherein said compound cocktail comprises, for example a TGFB receptor pathway inhibitor, for example, A-83-01, a glycogen synthase kinase-3 (GSK3) pathway inhibitor, for example, CHIR99021, FGF pathway stimulant, for example, FGF2, EGF pathway stimulant, for example, EGF, and VEGF pathway stimulant, for example, VEGF. In one aspect, the endoderm may comprise posterior foregut cells.

An endoderm population is intended to encompass one or more endoderm cells. Likewise, posterior foregut cells and a population thereof is intended to encompass one or more posterior foregut cells. Both endoderm cells and posterior foregut cells can be obtained using methods known in the art, for example, as described in Cheng, Xin et al. “Self-Renewing Endodermal Progenitor Lines Generated from Human Pluripotent Stem Cells.” Cell Stem Cell, Volume 10, Issue 4, 371-384; Hannan, Nicholas R. F. et al., “Generation of Multipotent Foregut Stem Cells from Human Pluripotent Stem Cells” Stem Cell Reports, Volume 1, Issue 4, 293-306; and Zhang, Ran-Ran et al. “Human iPSC-Derived Posterior Gut Progenitors Are Expandable and Capable of Forming Gut and Liver Organoids” Stem Cell Reports, Volume 10, Issue 3, 780-793

By clonal conditions, it is meant: conditions in which a culture of cells may be produced in such a way that an individual clone can be selected. For example, stem cells with multilineage differentiation potential and self-renewing capability may be clonally propagated in a clonal culture (clonal condition) to continuously produce hepatocytes and cholangiocytes as descendants while maintaining primitive stem cells, as described in Suzuki A, Zheng Y, Kaneko S, et al. Clonal identification and characterization of self-renewing pluripotent stem cells in the developing liver. The Journal of Cell Biology. 2002;156(1): 173-184. doi: 10.1083/jcb.200108066. In other aspects, the clonal culture (clonal condition) may include the feature of seeded cells with decreased density in culture as compared to what is typical in the art. For example, 1000-40000 cells embedded in 10 μl Matrigel will generate over 100-4000 of clone-derived organoids. Using this density, a single donor derived cells can be isolated, allowing for a single donor derived organoid. Clonal cell culture conditions are readily understood by one of ordinary skill in the art, and variations in protocol will be readily appreciated.

In one aspect, disclosed is a viable platform for screening a population phenotype in a pooled sample of various donor derived iPSCs. Human induced pluripotent stem cells (hiPSC) and other precursor cells provide a promising opportunity to establish human-specific models of various disease for drug development. Population-scale iPSC reprogramming initiatives may allow for the study of any imaginable population cohort for disease phenotypes, drug safety, and efficacy. However, one to one comparison of each individual stem cell is inefficient and completely unrealistic due to both time and cost considerations. The disclosed compositions and methods further allow for forward identification of rare individual phenotypes at the single cell level. Using the disclosed compositions and methods, a phenotype may be screened at a population scale in a single dish, enabling a new strategy to capture individual phenotype from a pooled sample—an approach termed “forward cellomics approach.” Such population scale hiPSC may provide an in vitro population cohort for drug safety and efficacy studies, thus addressing the aforementioned problems in the art.

Preclinical detection of risk compounds for drug induced liver injury (DILI) remain a significant challenge in drug development, highlighting a need for a predictive human system. Applicant has developed liver organoids from pooled iPSCs for analyzing humanistic DILI pathology at organoid resolution. Differentiated liver organoids from human iPSC contain polarized hepatocytes and an internal lumen lined by differentiated cells, recapitulating the in vivo bile canaliculi-like architecture. Applicant leveraged this structural feature for modeling DILI with multiplexed live imaging, termed “LoT” (Liver organoid-based Toxicity screen). LoT was functionally validated with 10 marketed drugs, distinguishing respective mechanism-of-action based on cholestasis and mitochondrial toxicity. (Data not shown.) Organoids were introduced in a vulnerable (steatotic) condition to demonstrate toxicity exaggeration as shown in clinics, followed by chemical rescue from massive organoid death. Thus, Applicant has shown the first human organoid-based model for drug safety with a cost-effective platform, facilitating compound optimization, mechanistic study, and precision medicine as anti-DILI therapy screening applications. Further, current phase 1 or other trial phases completely lack the risk assessment process of vulnerable conditions—the disclosed LoT methods allow use of a vulnerable (e.g., steatotic) condition to demonstrate toxicity exaggeration and addresses the huge financial loss that results from drug withdrawal by unpredictable hepatoxicity in current trials.

In one aspect, the disclosed methods and compositions allow for population-scale hiPSC reprogramming, which can then, in turn, allow for an in vitro population cohort for drug safety and efficacy studies. For example, a “healthy” or “disease” iPSC bank may be developed using the methods disclosed herein. For example, a single “iPSC bank” may comprise individual iPSCs that are derived from multiple donors. In some instances, the number of iPSC donor lines may be from about 100 iPSC donor lines up to over 1000 iPSC donor lines, or from more than one to about 500 donor lines. The instant disclosure provides methods by which the iPSC donor cells may be successfully synchronized and pooled to create a “pooled-iPSC” composition or “bank” of iPSC cells that are derived from more than one individual. The pooled-iPSCs may then be subjected to differentiation methods for the production of an organoid. The organoid may be, for example, a liver organoid (“mini-liver”) from the iPSC bank which, because of the ability to synchronize the iPSC cells, contain cells representative of a desired population.

In one aspect, liver organoids derived from the iPSC bank may be used for a variety purposes including drug screening and transplant application, Phase I studies, including hepatoxicity screening for both normal and compromised cells (e.g., steatotic liver cells/organoids), NAFLD/Cholestasis drug discovery screening, nutritional screening (such as, for example, TPN, supplements), or Phase II studies, nutrition and DILI Precision screening, mini-organ transplants, resilient plasma product generation, or as an FXR agonist (bile acid bearing intra-oral organoid delivery). In particular, bile acid or its analogue, is known as an FXR (farnesoid x receptor) agonist, which is currently used for treatment of NASH and other liver diseases. The disclosed organoids may be used for production of bile acids, which may be used for treatment of NASH and other such liver diseases. In other aspects, the organoid itself may be used in vivo as an FXR agonist when orally administered like compounds.

In one aspect, a method of testing a compound for a characteristic of interest is disclosed. The method may comprise the step of contacting a test composition of interest with the pooled organoid composition as described above composition. The characteristic of interest may be selected from cell death, cell growth, cell viability, cholestasis, mitochondrial overload, ROS production indicative of mitochondrial dysfunction, fibrosis, cell stiffness, fibrosis, efficacy for a disease state, and combinations thereof. The test compound may be selected from a drug-like molecule, a therapeutic agent, a nutritional supplement, or combinations thereof. In one aspect, the composition is a liver organoid having an induced or inherent disease state similar to or identical to NAFLD, cholestasis, and/or jaundice, and said characteristic of interest is efficacy for one or a plurality of such disease states.

In one aspect, a method of identifying a donor individual in a population is disclosed. The method may comprise the step of creating a representative pooled cell composition and identifying a cell that is an immunological match to a recipient in need of cells from said donor individual.

In one aspect, a method of identifying a genetic basis of a phenotype is disclosed. The method may comprise the steps of contacting an organoid or differentiated cell pool comprising cells derived from a pooled cell population derived from more than one donor; contacting said organoid or differentiated cell pool with a substance of interest, for example, a drug or drug-like substance (or any substance for which determining a cellular response is desired); assaying a phenotype of interest in said organoid or differentiated cell pool; and correlating said phenotype with a genotype from said pooled cell population.

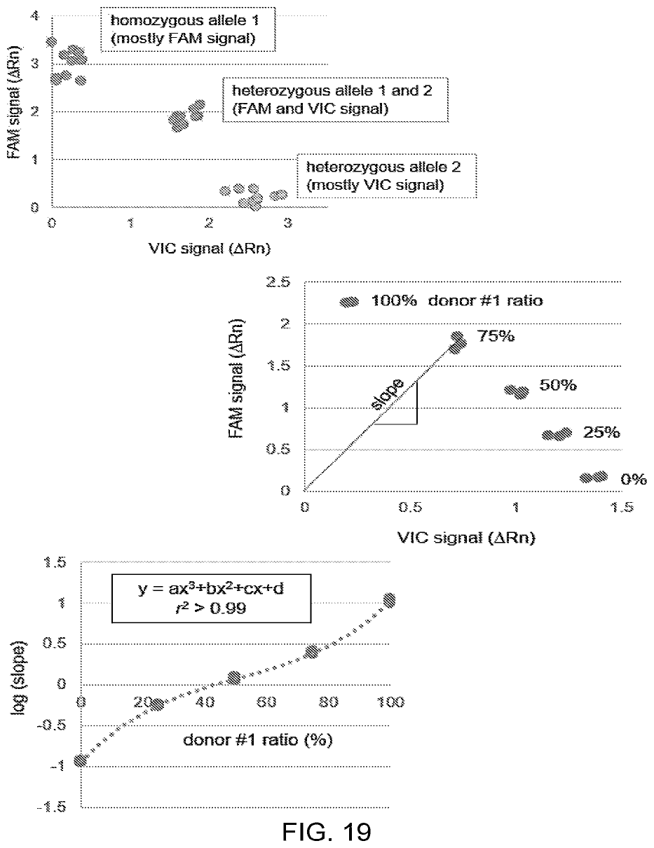

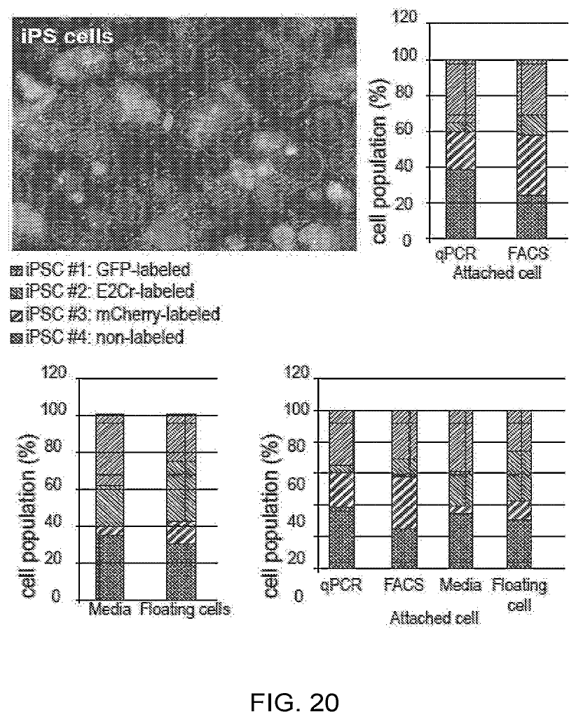

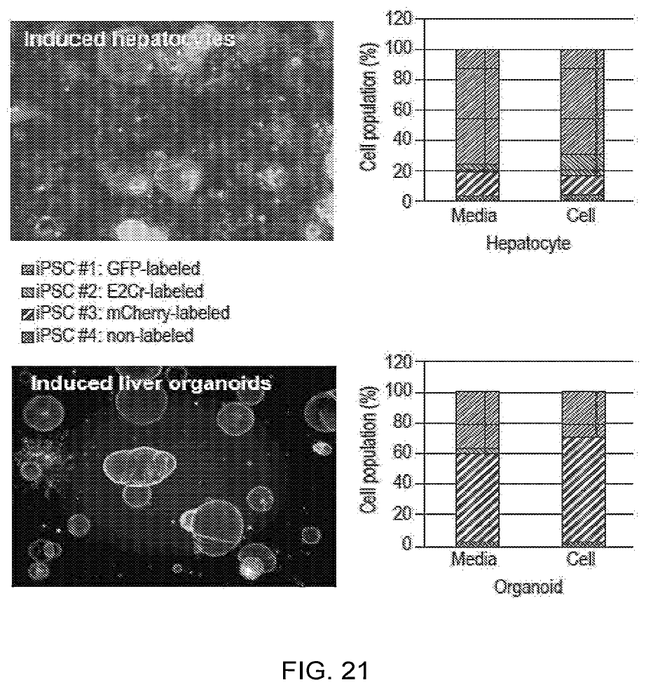

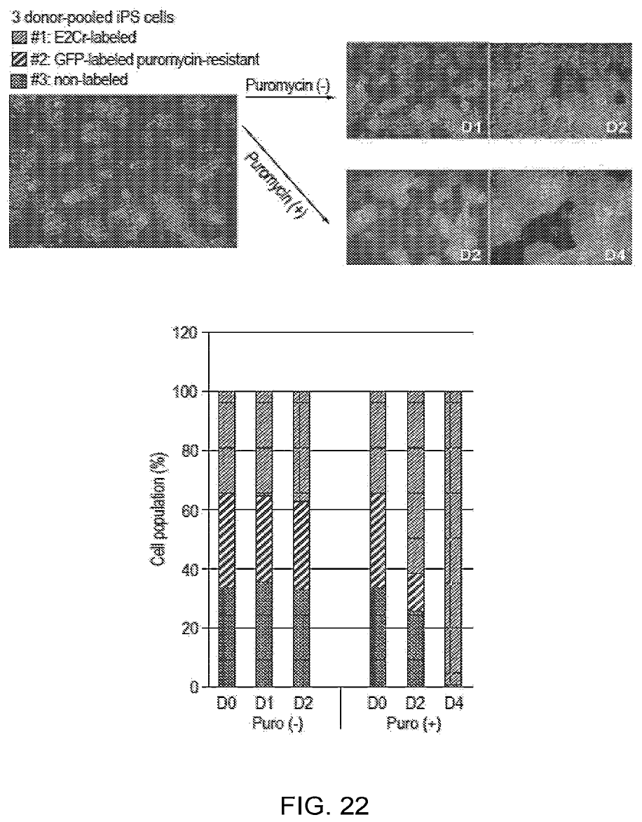

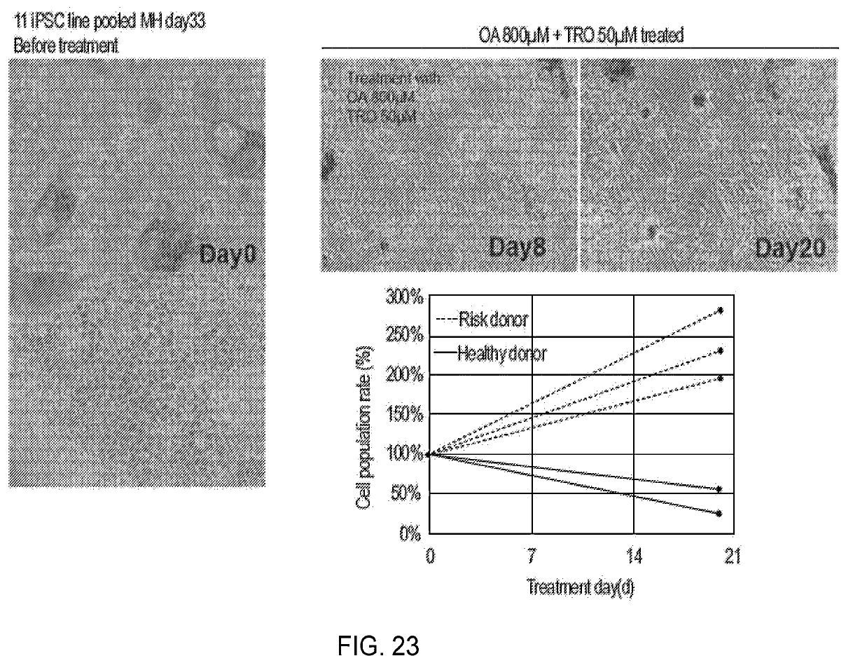

In one aspect, a “Drop out assay” is disclosed. In this aspect, genomic DNA in environments (environmental DNA, or “eDNA”), a form of cell-free DNA (cfDNA), exists in the environment to determine biodiversity such as sea and soil. Detection of circulating tumor derived cfDNA is an evolving component of cancer precision medicine. Applicant found that cells produce eDNA in a measurable amount in the cell culture supernatant. Remarkably, when applied into pooled stem cell culture, quantitative detection of respective donor correlates with the cell ratio in culture. Therefore, Applicant concluded eDNA-based noninvasive donor identification method is effective decoding strategy for pooled stem cell culture by eDNA in culture supernatant. PCR of eDNA in culture supernatant can be carried out using donor-specific SNP primers. Each SNP primer may be standardized, and the donor ratio of the iPSC pool can be estimated using eDNA contained in the culture supernatant (see FIG. 19). As a result, the cell ratio may be successfully determined by PCR of eDNA in the medium (see FIG. 20). In addition, these methodologies may be applied into differentiated progeny such as hepatocyte and liver organoid derived from iPSC pool (see FIG. 21). In order to confirm the sensitivity of eDNA donor identification, Applicant monitored cell ratio change by drug selection. Three donors—two fluorescently labeled donors and one puromycin resistant donor—were mixed and cultured in a puromycin containing media. As a result of eDNA based donor detection, puromycin resistant cells dominated over time, whereas two other donors died due to puromycin toxicity. These results were further confirmed by fluorescent imaging in parallel, suggesting specific donor drop out can be measurable by eDNA method, hereafter named ‘drop out assay’ (see FIG. 22). Finally, as a proof-of-principle drug screening assay, hepatotoxic drug treatment was performed using fat-treated hepatocytes prepared from 11 iPSCs pool (see FIG. 23). eDNA based drop-out assay confirmed cells with high fat accumulation risk donor were significantly more sensitive to drug induced hepatotoxicity than healthy donor derived cells (FIG. 23).

In one aspect, “Adaptive immune-matching” is disclosed. In this aspect, a pool of a representative population of iPSCs to create a “panel” can be made. At least some of the pools may be an immunological match to potential patient (recipient). The approach may be used to waive the prior matching of stem cell to recipient, thus reducing the labor-intensive collection of stem cell library as well as cost associated with banking.

In one aspect, a method of identifying a genetic basis of a phenotype is disclosed. The method may comprise the steps of developing an organoid from pooled patients, exposing substances of interests such as drug, analyzing individual response (phenotype) in a pool, and re-analyzing original genome data to match up the phenotype to know the genomic contribution.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Synchronization of Growth Kinetic Variabilities Among Multiple iPSCs

Applicant has shown that hypoxic conditions can be used to synchronize growth kinetic variabilities. Hypoxic culture methods enabled successful growth synchronization. Applicant has successfully identified synchronizing factors including oxygen and inhibitor treatments. For example, cell growth in the presence and absence of an estrogen-related receptor gamma agonist (GSK5182), a mitogen-activated protein kinase inhibitor (PD0325901), a glycogen synthase kinase-3 inhibitor (CHIR99021), a Rho-associated kinase inhibitor (Y-27632) and a type 1 transforming growth factor-b receptor inhibitor (A-83-01), in ambient and hypoxic conditions may be used to synchronize cells from multiple donors. In one aspect, a hypoxic culture method of 2% O2 conditions may be used to enable successful growth synchronization and maintenance in each cell population.

### Expansion and Differentiation of Stem Cell Pool into Specific Cell Types

Applicant has further demonstrated co-differentiation induction of various cell types from iPSC pool. DE, mesoderm, posterior foregut, endothelia induction (and tube formation), hepatocyte induction, liver organoid induction, showing that pooled cells are competent for multi-lineage differentiation, and that iPSC pooling overcomes differentiation propensity to endothelial cells.

An iPSC pool with a large population which may then be used to decode the individuals at single cell level. For example, 10-15 cell lines pool may be differentiated into liver organoids, and both genomic and cDNA may be sequenced. Analyzed at single cell with Drop-seq (a large-scale single cell sequencing method), donor difference effect and rare population effect may be detected. Such stem cell synchronization approach may provide an unprecedented powerful approach for evaluating a population phenotype in a dish, allowing for conducting clinical trials in a dish and promoting drug development process as well as precision medicine.

### “Human Organoid Trial” (HoT) in a Dish

Population pooling into an iPSC-panel, for example, Asian, American, and African populations, can be pooled into an iPSC-organoid panel. Using multi-plexed high-content imaging, various cell parameters may be measured. Such parameters include, for example, cell death, cholestasis, mitochondrial overload, and ROS production. Such parameters may be assessed in response to one or more compounds of interest. The system may be used for drug development including efficacy, safety, transplant therapy, and adaptive immune-matching.

### Analysis of the Donor-Dependent Variation

Cell proliferation and metabolism to examine donor dependence of each cell may be analyzed. Cell proliferation and metabolism of each iPSC may be very different, as determined by NMR based metabolomics data and cell proliferation data.

Screening of cell synchronized condition. 1) first, growth kinetic variables may be synchronized by hypoxic culture conditions (about 2% to about 5% O2) by regulating cell growth kinetics. 2) compound cocktail may be used to promote iPSC synchronization. The compound cocktail treatment may support cell synchronization by combining with the hypoxic culture. The SYNDEX (synchronization index) can be used to show the reciprocal of donor difference standard deviation.

Pooling of multi-donor iPSC. Hypoxic conditions may enable synchronization and maintenance in each cell population on same dish.

Detection of the rare phenotype in iPSC pool. In one aspect, a rare phenotype may be detected in an iPSC pooled composition. For example, for detection of hemochromatosis, iPSCs from individual 1 and individual 2 and individual 3 may be pooled and subjected to co-differentiation to form a liver organoid according to methods known thin the art. Iron accumulation in the resulting liver organoid may be detected by live imaging. The hemochromatosis phenotype could then be distinguished from pooled organoids in the accumulated state of iron. the hemochromatosis population can be distinguished from the pooled organoid. Thus, the methods may be useful for detecting the influence of a rare disease effect in a pooled sample.

### Maintenance of PSCs

TkDA3 human iPSC clone used in this study was kindly provided by K. Eto and H. Nakauchi. The TkDA3 human iPSC clone used in this study was kindly provided by K. Eto and H. Nakauchi. 1231A3, 317D6, 1383D6, and FF01 were gifted by Kyoto University (Japan). CW027, CW077, CW150 and WD90, 91, 92 were purchased from Coriell (NJ, USA). Human iPSC lines were maintained as described previously (Takebe et al., 2015; Takebe et al., 2014). Undifferentiated hiPSCs were maintained on feeder-free conditions in mTeSR1 medium (StemCell technologies, Vancouver, Canada) on plates coated with Matrigel (Corning Inc., NY, USA) at 1/30 dilution at 37° C. in 5% CO₂. Culture oxygen conditions were performed with 2%, 5% and ambient (20%) using multi-gas incubator MCO-19 MUV (Panasonic, Japan).

Inhibitor Treatment for Synchronization of PSCs

After being cultured in a 6 multi-well plate, cells were treated with inhibitors for growth synchronization. The following antibodies were used: GSK5182 (agonist for estrogen-related receptor γ), PD0325901 (mitogen-activated protein kinase inhibitor), CHIR99021 (glycogen synthase kinase-3 inhibitor), Y-27632 (Rho-associated kinase inhibitor) and A-83-01 (type 1 transforming growth factor-b receptor inhibitor).

Differentiation of PSCs into Definitive Endoderm

Differentiation of hiPSCs into definitive endoderm was induced using previously described methods with slight modifications (Spence et al., 2011). In brief, colonies of hiPSCs were isolated in Accutase (Thermo Fisher Scientific Inc., Waltham, MA, USA) and 150000 cells/mL were plated on Matrigel coated tissue culture plate (VWR Scientific Products, West Chester, PA). Medium was changed to RPMI 1640 medium (Life Technologies, Carlsbad, CA) containing 100 ng/mL Activin A (R&D Systems, Minneapolis, MN) and 50 ng/mL bone morphogenetic protein 4 (BMP4; R&D Systems) at Day 1, 100 ng/mL Activin A and 0.2% fetal calf serum (FCS; Thermo Fisher Scientific Inc.) at Day 2 and 100 ng/mL Activin A and 2% FCS at Day 3. For Day 4-6, cells were cultured in Advanced DMEM/F12 (Thermo Fisher Scientific Inc.) with B27 (Life Technologies) and N2 (Gibco, Rockville, MD) containing 500 ng/ml fibroblast growth factor (FGF4; R&D Systems) and 3 μM CHIR99021 (Stemgent, Cambridge, MA, USA). Cultures for cell differentiation were maintained at 37° C. in an atmosphere of 5% CO2/95% air and the medium was replaced every day. Differentiated definitive endoderm showed budding on the plate at Day 7.

### Freezing Posterior Foregut (pFG) Cells

Carefully aspirate media from the cell culture flask. (Rinsing with PBS is not necessary). Immediately add Accutase to the flask in an amount sufficient to cover the cells (typically 1 mL/well for a 6 well plate). Set the flask in 37° C. incubator for 5-6 min. Check the flask to see if cells have rounded. Smack the flask against the palm of your hand to dislodge any “stickers”. Gently disperse the cells. Add wash media and take a sample to count cells. Centrifuge for 3 min @ 200-300×g. Aspirate supernatant. Add mTESR1+Rock inhibitor (10 μM). Plate cells (2-5×10⁴ for maintenance). Passaging pFG cells. Label freezing vials with cell line, cell number, date, freezer medium, growing medium, and plate coating (laminin or Matrigel). Aspirate supernatant. Add 500 μL Cell Banker1 to 0.5-2×10⁶ cells. Add vial to Nalgene Cryo Freezing container for 24 hr. Store at −80°C., or transfer to liquid nitrogen for long-term storage.

### Lipids and Iron Live-Cell Imaging

After being cultured in an ultra-low attachment 6 multi-well plate, 5-10 HLO were picked up and seeded in a Microslide 8 Well Glass Bottom plate (Ibidi, WI, USA) and subjected to live-cell staining. The following antibodies were used: BODIPY® 493/503 for lipids (Thermo Fisher Scientific Inc.) and FeRhoNox-1 546 for iron accumulation (Goryokagaku Inc.) Nuclear staining was marked by NucBlue Live ReadyProbes Reagent (Thermo Fisher Scientific Inc.). HLO was visualized and scanned on the KEYENCE BZ-X710 Fluorescence Microscope (Japan). The final lipid droplet volume was calculated by IMARIS8 and normalized by each organoid size.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising a differentiated cell population, wherein said differentiated cell population comprises cells from more than one individual.

2. The composition of claim 1, wherein said differentiated cell population is selected from definitive endoderm, mesoderm, exoderm, posterior foregut, endothelium, hepatocytes, or an organoid.

3. The composition of claim 1 or 2, wherein said more than one individual comprises a population of individuals having a disease of interest.

4. The composition of any preceding claim, wherein said differentiated cell population comprises an organoid selected from a liver organoid, a gastric organoid, an intestinal organoid, a brain organoid, a pulmonary organoid, a bone organoid, a cartilage organoid, a bladder organoid, a blood vessel organoid, an endocrine organoid, a sensory organoid.

5. A method of making a composition comprising a differentiated cell population, wherein said a differentiated cell population comprises a population of cells derived from more than one individual, for example, from about 10 individuals to about 1,000 donors comprising the step of exposing a pooled-precursor cell population, preferably a cell type selected from embryonic stem cells (ESC), embryonic carcinoma cells (ECs), epiblast stem cells (EpiSC), differentiated posterior foregut cells, or combinations thereof, to a synchronization condition for a period of time sufficient to produce a synchronized pooled-precursor cell population;
wherein said synchronization condition comprises a compound cocktail comprising a MEK/ERK Pathway Inhibitor, preferably a mitogen-activated protein kinase inhibitor, more preferably PD0325901, and a glycogen synthase kinase-3 (GSK3) pathway inhibitor, preferably CHIR99021.

6. The method of claim 5, wherein said cocktail further comprises an estrogen-related receptor gamma (ERRγ) agonist, preferably GSK5182, a RHO/ROCK pathway inhibitor, preferably a Rho-associated kinase inhibitor, preferably Y-27632 and a type 1 transforming growth factor-b (TGFB) receptor pathway inhibitor, preferably A-83-01.

7. The method of claim 5 or 6, wherein said synchronization condition comprises a hypoxic culture condition, preferably wherein said hypoxic culture condition is between about 2% O₂ to about 5% O₂.

8. The method of any of claims 5 through 7, further comprising the step of culturing said synchronized pooled-precursor cell population for a period of time and under conditions sufficient to differentiate said precursor cells into definitive endoderm.

9. The method of claim 8, wherein said definitive endoderm is further differentiated into an organoid composition, wherein said organoid composition is characterized by comprising cells from more than one donor.

10. A method of making a composition comprising a differentiated cell population from more than one donor, preferably from about 10 donors to about 1,000 donors comprising the steps of
a. exposing a precursor cell population to a clonal condition for a period of time sufficient to produce a synchronized pooled-endoderm population;
b. contacting said pooled-endoderm cell population with a compound cocktail, wherein said compound cocktail comprises, for example a TGFB receptor pathway inhibitor, preferably A-83-01, a glycogen synthase kinase-3 (GSK3) pathway inhibitor, preferably CHIR99021, FGF pathway stimulant, preferably FGF2, EGF pathway stimulant, preferably EGF, and VEGF pathway stimulant, preferably VEGF;
preferably wherein said step a and/or step b are carried out in a hypoxic culture condition of between about 2% O₂ to about 5% O₂.

11. The method of claim 10, wherein said endoderm comprises posterior foregut cells.

12. A method of testing a compound for a characteristic of interest, comprising the step of contacting a test composition of interest with the composition of any of claims 1 through 4.

13. The method of claim 12, wherein said characteristic of interest is selected from cell death, cell growth, cell viability, cholestasis, mitochondrial overload, ROS production indicative of mitochondrial dysfunction, fibrosis, cell stiffness, fibrosis, efficacy for a disease state, and combinations thereof.

14. The method of claim 12 or 13, wherein said test composition is selected from a drug-like molecule, a therapeutic agent, a nutritional supplement, or combinations thereof.

15. The method of any of claims 12 through 14, wherein said composition is a liver organoid and said characteristic of interest is efficacy for NAFLD/cholestasis/jaundice, wherein a disease state is induced in said liver organoid.

16. A method of identifying a donor individual in a population, comprising creating a representative pooled cell composition, and identifying a cell that is an immunological match to a recipient in need of cells from said donor individual.

17. A method of identifying a genetic basis of a phenotype, comprising the steps of
a. contacting an organoid or differentiated cell pool comprising cells derived from a pooled cell population derived from more than one donor;
b. contacting said organoid or differentiated cell pool with a substance of interest, preferably a drug or drug-like substance;
c. assaying a phenotype of interest in said organoid or differentiated cell pool; and
d. correlating said phenotype with a genotype from said pooled cell population.
